(19) United States
(12) Patent Application Publication
(10) Pub. No.: US 2010/0087407 A1
(43) Pub. Date: Apr. 8, 2010

(54) USE OF AROMATASE INHIBITORS

(76) Inventor: James Symons, Ann Arbor, MI (US)

Correspondence Address: Jonathan P. O''Brien, Ph.D.;Honigman Miller Schwartz and Cohn, 350 East Michigan Avenue, Suite 300, KALAMAZOO, MI, 49007, US

(21) Appl. No.: 12/309,957

(22) PCT Filed: Aug. 3, 2007
(86) PCT No.: PCT/US2007/017317

§ 371 Date: May 28, 2009

Related U.S. Application Data
(60) Provisional application No. 60/835,825, filed on Aug. 4, 2006.

(51) Int. Cl. A61K 31/56; A61K 31/4196

(52) U.S. Cl. 514/171; 514/383; 514/177

(57) ABSTRACT

The invention provides a method of thinning the endometrium in a woman by utilizing a therapeutically effective amount of an aromatase inhibitor.

### CROSS REFERENCE TO RELATED APPLICATIONS

This application claims priority to U.S. Ser. No. 60/835,825, filed Aug. 4, 2006. The entire content of the aforementioned application is incorporated herein.

### BACKGROUND

Excessive uterine bleeding, menorrhagia, is one of the most common reasons for gynecology referral in pre-menopausal women. Although medical therapy is generally the first approach, many women will eventually require a hysterectomy. Hysterectomy is associated with a significant in-patient hospital stay and a period of convalescence that makes it an unattractive and unnecessarily invasive option for many women. Hysteroscopic endometrial ablation or resection, and more recently “second generation” devices such as balloon or microwave ablation offer a day-case surgical alternative to hysterectomy for these women. These methods are also cheaper procedures than hysterectomy. Hysterectomy guarantees amenorrhea, but is costly and has a significant impact on health-related quality of life immediately after surgery.

Complete endometrial removal or destruction is one of the most important determinants of treatment success. Therefore endometrial ablation will be most effective if undertaken in the immediate post-menstrual phase when endometrial thickness is usually less than four mm, the thickness at which most methods of endometrial ablation are effective in destroying. However there are often difficulties in reliably arranging surgery for this time.

Human endometrium is a unique tissue that undergoes sequential phases of proliferation, and secretory changes followed by tissue shedding and bleeding during menstruation. Proliferation of the endometrial cells occurs in response to estrogen stimulation particularly during the first half of the menstrual cycle (follicular or proliferative phase). Menstruation is the process by which the endometrium is discarded each month if pregnancy fails to occur. It involves sloughing of the endometrium over a period of days, bleeding and subsequent repair. Work carried out in the 1930s established that ovarian steroids, estrogen and progesterone, were responsible for the changes in endometrial structure and function throughout the cycle.

Within the uterus, the female sex steroids estrogen and progesterone play pivotal roles in endometrial development. More specifically, these steroids regulate a multitude of cellular processes, which include cell proliferation and differentiation, as well as regulation of vascular permeability, angiogenesis and adenogenesis. To bring about these changes, estrogen and progesterone must appropriately modulate a variety of factors, which include growth factors, cytokines, extracellular matrix proteins and adhesion molecules.

Steroids interact with their target organs via specific nuclear receptors. The expression of endometrial sex steroid receptors (progesterone receptor (PR), estrogen receptor (ER), androgen receptor (AR), all of which are nuclear proteins, varies both temporally and spatially across the menstrual cycle. The expression of ER and PR are under dual control of estrogen and progesterone. Both endometrial ER and PR are up-regulated during the follicular phase by ovarian estrogen and subsequently down regulated in the luteal phase by progesterone acting at both the transcriptional and the post-transcriptional levels. Experiments with rhesus macaques that have been treated with estrogen and progesterone indicate that the induction of menstruation is identical under the following two conditions: withdrawal of progesterone alone while estrogen is maintained, or withdrawal of both estrogen and progesterone. Furthermore, the administration of the antiprogestin, mifepristone (RU486) is associated with marked endometrial extracellular matrix (ECM) breakdown and excessive menstrual bleeding.

During the menstrual cycle endometrial thickness varies from as little as one mm in the immediate postmenstrual phase to ten mm or more in the late secretary phase. The radius of a standard electrosurgery loop used for endometrial resection is about four mm and the depth of tissue destruction with Nd:YAG laser or a roller ball electrode is four to six mm. With these depths of tissue removal or destruction, it is apparent that surgery will be most effective if undertaken when endometrial thickness is less than 4 mm, either in the immediate post-menstrual phase or following the administration of hormonal agents which induce endometrial thinning or atrophy. Second generation endometrial ablation devices which are now being more broadly deployed in the gynecology specialty community often have relied on pretreatment with a GnRH agonist or endometrial aspiration prior to the ablation procedure in completing their clinical studies to support patient outcomes in the medical device approval trials reviewed by the FDA.

The proportion of women who experience amenorrhea following endometrial ablation varies in different series from 30 to 60%; though the proportion experiencing an improvement in menstrual symptoms is considerably higher.

Difficulty in reliably arranging surgery in the immediate post-menstrual phase and the unpredictable thickness of the unprepared endometrium has resulted in much attention being given to the use of endometrial thinning agents prior to surgery. A number of randomized studies have now been undertaken comparing different hormonal agents with each other or with no pre-operative treatment or placebo. The most commonly evaluated agents have been gonadotropin-releasing hormone (GnRH) agonists and danazol. Progestogens have also been studied.

It has been suggested that the use of these agents, particularly GnRH analogues, will reduce operating time, improve the intra-uterine operating environment, and reduce distension medium absorption. It is also possible that their use may also improve postoperative outcome. An improved operating environment might also reduce the rate of complications associated with these procedures. An improvement in post-operative outcome might increase patient satisfaction and reduce the proportion of women undergoing subsequent hysterectomy. However, these agents do add significant cost to any hysteroscopic procedure. Administration timing of 1-2 months prior to the procedure, coupled with their side-effect profile, makes these alternatives sub-optimal.

Cost would be one factor that would make the use of progestogens attractive, as they are significantly cheaper than both GnRH analogues and danazol. In a small, randomized study evaluating the effect of progestogens on endometrial thickness alone, norethisterone and medroxyprogesterone acetate had no effect on endometrial thickness, though cyproterone acetate did produce a significant reduction in endometrial thickness in an amount similar to danazol. Observational studies that have included patients treated with different progestogens have reported disappointing effects.

Endometrial thinning prior to hysteroscopic surgery in the early proliferative phase of the menstrual cycle for treating menorrhagia improves both the operating conditions for the surgeon and short-term post-operative outcome. Gonadotropin-releasing hormone analogues produce slightly more consistent endometrial thinning than danazol, though both agents produce satisfactory results. The effect of these agents on longer-term post-operative outcomes such as amenorrhea and the need for further surgical intervention reduces with time.

There is, therefore, a need for less expensive agents for endometrial thinning prior to endometrial ablation or other surgical procedures on the endometrial cavity and uterus.

### SUMMARY OF THE INVENTION

In one aspect, the present invention relates to a method of thinning the endometrium in a woman, which includes administering to a woman in need thereof a therapeutically effective amount of an aromatase inhibitor.

In another aspect, the present invention relates to a method of preparing a woman for surgical ablation of the endometrium, which includes administering to the woman in need thereof a therapeutically effective amount of an aromatase inhibitor.

In still another aspect, the present invention relates to a method of treating menorrhagia in a woman, which includes administering to the woman in need thereof a therapeutically effective amount of an aromatase inhibitor.

In some embodiments, the aromatase inhibitor can be administered to the woman at any time of the woman's menstrual cycle.

In some other embodiments, the aromatase inhibitor is first administered on the 2^{nd}, 3^{rd}, 4^{th}, or 5^{th} day of the cycle.

In still some other embodiments, the aromatase inhibitor is first administered on the 6^{th}, 7^{th}, 8^{th}, or 9^{th} day of the cycle.

In still some other embodiments, the aromatase inhibitor is first administered on the 10^{th}, 11^{th}, 12^{th}, or 13^{th} day of the cycle.

In still some other embodiments, the aromatase inhibitor is first administered on the 14^{th}, 15^{th}, 16^{th}, or 17^{th} day of the cycle.

In still some other embodiments, the aromatase inhibitor is first administered on the 18^{th}, 19^{th}, 20^{th}, or 21^{st} day of the cycle.

In some embodiments, the aromatase inhibitor is administered once everyday for 2 to 10 (e.g., 3 to 7) consecutive days.

In some other embodiments, about 1 to 300 mg (e.g., about 1 to 5 mg, about 1 to 15 mg, about 5 to 30 mg, about 7 to 20 mg, about 30 to 300 mg, about 60 to 150 mg, about 1 to 150 mg, about 15 to 150 mg, about 30 to 150 mg, about 5 mg, about 10 mg, about 60 mg, about 120 mg, about 150 mg, about 300 mg) of the aromatase inhibitor is administered on the first day.

In still some other embodiments, about 1 to 150 mg (e.g., about 1 to 5 mg, about 1 to 15 mg, about 15 to 150 mg, about 30 to 150 mg, about 5 mg, about 30 mg, about 60 mg, or about 150 mg) of the aromatase inhibitor is administered everyday after the first day.

In some embodiments, the aromatase inhibitor is anastrozole, letrozole, exemestane, or a combination thereof.

In still further embodiments, about 1 to about 120 mg of anastrozole or about 60 letrozole is administered on the first day.

In other aspects, about 1 to about 120 mg of anastrozole or up to about 300 letrozole is administered on the first day.

In further embodiments, about 60 mg of anastrozole or about 30 mg of letrozole is administered everyday after the first day.

In still further embodiments, about 60 mg of anastrozole or up to about 150 mg of letrozole is administered everyday after the first day.

Also within the scope of this invention is a method for maintaining the thinness of endometrium in a woman by administering to the woman in need thereof a therapeutically effective amount of an aromatase inhibitor. The aromatase inhibitor can be administered, e.g., in the early phase of the menstrual cycle (e.g., any of the first 10 days of the cycle) in this woman. The aromatase inhibitor and the dosing amount and frequency can be the same or different from those provided above.

### DETAILED DESCRIPTION OF THE INVENTION

As used herein, the term “aromatase inhibitors” refers to any compounds that are capable of inhibiting the enzyme aromatase. Aromatase inhibitors may have a non-steroidal or a steroidal chemical structure. Both non-steroidal aromatase inhibitors and steroidal aromatase inhibitors can be used for this invention. Examples of aromatase inhibitors include aminoglutethimide, anastrozole (Arimidex®), letrozole (Femara®), exemestane (Aromasin®), and 4-androstene-3,6,17-trione. Additional examples of aromatase inhibitors are described in British Patent Application No. GB-A-2 17 1100, European Patent Publication Nos. EP-A 165 904, EP-A 281 283, EP-A 296 749, EP-A 299 684, EP-A 236 940, EP-A 408 509, EP-A 316 097, EP-A 354 689, EP-A 354 683, EP-A 181 287, EP-A 337 928, EP-A 340 153, EP-A 114 033, EP-A 166 692, and EP-A 337 929; Swiss Patent Application Nos. 1339/90-7, 3014/90-0, 3014/90-0, and 3923/90-4; German Patent Application Nos. DE-A 4 014 006, DE-A 3 926 365, and DE-A 3 740 125; U.S. Pat. No. 4,322,416, and U.S. Provisional Application Ser. No. 60/615,978. All of the publications cited herein are incorporated by reference in their entireties.

The in vitro inhibition of aromatase activity can be demonstrated, for example, using the methods described in J. Biol. Chem., Vol. 249, page 5364 (1974) or in J. Enzyme Inhib., Vol. 4, page 169 (1990). In addition, IC₅₀ values for aromatase inhibition can be obtained, for example, in vitro by a direct product isolation method relating to inhibition of the conversion of 4-¹⁴C-androstenedione to 4-¹⁴C-oestrone in human placental microsomes.

The In vivo aromatase inhibition can be determined, for example, by the following method as described in J. Enzyme Inhib., 1990, 4, 179.

As used herein, a “woman” can be a healthy woman or a female patient with medical conditions. Examples of the related medical conditions are those related to a woman's reproductive system, e.g., menorrhagia. Both pre-menopausal women and post-menopausal women are suitable subjects of the present invention.

As used herein, the term “menstrual cycle” refers to a recurring cycle of physiological changes in the females of some animal species that is associated with reproductive fertility. While the cycle length may vary from woman to woman, 28 days is generally taken as representative of the average ovulatory cycle in women. For the purpose of this invention, the onset of menstrual bleeding marks the beginning of the cycle, so the first day of bleeding is also referred to as the first day of the cycle.

As used herein, a “therapeutically effective amount” is defined as the amount required to confer a therapeutic effect on the treated subject (i.e., a woman), and is typically determined based on age, surface area, weight, and condition of the subject. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970).

Aromatase

Aromatase is a microsomal member of the cytochrome P450 hemoprotein containing enzyme complex superfamily (P450arom, the product of the CYP19 gene) that catalyzes the rate-limiting step in the production of estrogens, that is, the conversion of androstenedione and testosterone via three hydroxylation steps to estrone and estradiol respectively. Aromatase activity is present in many tissues, such as the ovaries, the brain, adipose tissue, muscle, liver, breast tissue, and in malignant breast tumors. The main sources of circulating estrogens are the ovaries in premenopausal women and adipose tissue in postmenopausal women.

Aromatase is a good target for selective inhibition because estrogen production is a terminal step in the biosynthetic sequence. A large number of aromatase inhibitors have been developed and utilized in clinical studies over the last 20 years, mainly for treatment of breast cancer.

The first aromatase inhibitor to be used clinically was aminoglutethimide, which induces a medical adrenalectomy by inhibiting many other enzymes involved in steroid biosynthesis. Although aminoglutethimide is an effective hormonal agent in postmenopausal breast cancer, its use is complicated by the need for concurrent corticosteroid replacement. In addition side effects, like lethargy, rashes, nausea and fever, result in 8-15% of patients stopping the aminoglutethimide treatment. The lack of specificity and unfavorable toxicity profile of aminoglutethimide has led to a search for more specific aromatase inhibitors. In addition, the earlier aromatase inhibitors were not able to completely inhibit aromatase activity in premenopausal patients. Therefore, aromatase inhibitors have been primarily used for postmenopausal patients.

Aromatase inhibitors have been classified in a number of different ways, including first-, second-, and third-generation; steroidal and nonsteroidal; and by binding activity, i.e., reversible (ionic binding) and irreversible (suicide inhibitor, covalent binding). The most successful, third generation aromatase inhibitors are now available commercially for breast cancer treatment.

The commercially available agents include two nonsteroidal preparations, anastrozole and letrozole, and a steroidal agent, exemestane. Exemestane is available from Pfizer Inc., New York, N.Y. under the trademark Aromasin®; anastrozole, is available from AstraZeneca under the trademark Arimidex® (ZN 1033); and letrozole is available from Novartis Pharmaceutical Corporation under the trademark Femara® CGS 20267). Anastrozole and letrozole are selective aromatase inhibitors, available for clinical use in North America, Europe and other parts of the world for treatment of postmenopausal breast cancer. These triazole derivatives are reversible, competitive aromatase inhibitors, which are highly potent and selective. Their intrinsic potency is considerably greater than that of aminoglutethimide, and at doses of 1-5 mg/day, they inhibit estrogen levels by 97% to >99% in post-menopausal women. This level of aromatase inhibition results in estradiol concentrations below detection by most sensitive immunoassays. The high affinity of aromatase inhibitors for aromatase is thought to reside in the −4 nitrogen of the triazole ring that coordinates with the heme iron atom of the aromatase enzyme complex. Aromatase inhibitors are completely absorbed after oral administration with mean terminal t_{1/2} of approximately 45 hr (range, 30-60 hr). They are cleared from the systemic circulation mainly by the liver. Gastrointestinal disturbances account for most of the adverse events, although these have seldom limited therapy. Other adverse effects are asthenia, hot flashes, headache, and back pain.

The wide clinical safety of aromatase inhibitors, as well as the reduced cost of treatment, makes these agents promising for use in treatment modalities for estrogen dependent disorders, e.g., endometriosis and uterine fibroids. Although these agents are mainly used in postmenopausal women, most recently, the success of these agents in inhibiting estrogen production in women of the reproductive age group has been demonstrated. See, e.g., Mitwally, et al., Aromatase Inhibition: A novel method of ovulation induction in women with polycystic ovarian syndrome, Reprod. Technol., Vol. 10, No. 5, pages 244-247 (2000); Mitwally, et al., Use of an aromatase inhibitor for induction of ovulation in patients with an inadequate response to clomiphene citrate, Fertil. Steril., Vol. 75, No. 2, 305-9 (2001); and Mitwally, et al., Aromatase inhibition improves ovarian response to FSH: A potential option for low responders during ovarian stimulation, Fertil. Steril., Vol. 77, No. 4, pages 776-80 (2002).

Without wishing to be bound by theory, an aromatase inhibitor may be used to thin the endometrium by decreasing estrogen production during the pre-operative period before endometrial ablation or used to treat menorrhagia.

Expression of the aromatase enzyme found in the endometrium is associated with different estrogen disorders such as endometriosis, uterine fibroids, or menorrhagia. Estrogen produced by local aromatase activity can exert its effects by readily binding to its nuclear receptor within the same cell. Disease-free endometrium and myometrium, on the other hand, lack aromatase expression.

In general, aromatase inhibitors known in the art are usually suitable for practicing the present invention. Examples of aromatase inhibitors have been provided above. Some of them, e.g., anastrozole, letrozole, and exemestane, may be commercially available, either as a pure compound or in the form of a pharmaceutical composition (e.g., pills for treating breast cancer), while most of them generally can be prepared by methods known in the art. Suitable methods have been provided in the publications identified above, which are incorporated herein by reference in their entireties.

Compositions and Delivery

Aromatase inhibitors can be used to practice the present invention in the same way as most other types of compounds for their respective pharmaceutical applications. They can be formulated, together with a pharmaceutically acceptable carrier, adjuvant, vehicle, or excipient, into pharmaceutical compositions for various routes of administration, e.g., for enteral, such as peroral or rectal administration, also for transdermal or sublingual administration, and for parenteral, for example intravenous, subcutaneous and intramuscular, administration.

The proportion of active ingredient in such pharmaceutical compositions is generally from approximately 0.001% to approximately 60%, e.g., from approximately 0.1% to approximately 20%.

Suitable excipients for pharmaceutical compositions for oral administration are, e.g., fillers, such as sugars, for example lactose, saccharose, mannitol or sorbitol, cellulose preparations and/or calcium phosphates, for example tricalcium phosphate or calcium hydrogen phosphate, and binders, such as starches, for example corn, wheat, rice or potato starch, gelatin, tragacanth, methylcellulose and/or hydroxypropylcellulose, disintegrators, such as the above-mentioned starches, also carboxymethyl starch, crosslinked polyvinylpyrrolidone, agar, alginic acid or a salt thereof, such as sodium alginate, and/or cellulose, for example in the form of crystals, especially in the form of microcrystals, and/or flow regulators and lubricants, for example silicic acid, talc, stearic acid or salts thereof, such as magnesium or calcium stearate, cellulose and/or polyethylene glycol.

Examples of orally administrable pharmaceutical compositions are dry-filled capsules consisting of gelatin, and also soft sealed capsules consisting of gelatin and a plasticiser, such as glycerol or sorbitol. The dry-filled capsules may contain the active ingredient in the form of granules, for example in admixture with fillers, such as lactose, binders, such as starches, and/or glidants, such as talc or magnesium stearate, and, if desired, stabilisers. In soft capsules, the active ingredient is preferably dissolved or suspended in suitable oily excipients, such as fatty oils, paraffin oil or liquid polyethylene glycols, to which stabilisers and/or anti-bacterial agents may also be added. There may also be used capsules that are easily bitten through, in order to achieve by means of the sublingual ingestion of the active ingredient that takes place as rapid an action as possible.

Examples of rectally or transvaginally administrable pharmaceutical compositions are, for example, suppositories that consist of a combination of the active ingredient with a suppository base. Suitable suppository bases are, for example, natural or synthetic triglycerides, paraffin hydrocarbons, polyethylene glycols or higher alkanols. There may also be used gelatin rectal capsules, which contain a combination of the active ingredient with a base material. Suitable base materials are, for example, liquid triglycerides, polyethylene glycols or paraffin hydrocarbons.

Examples of formulations for transdermal administration comprise the active ingredient together with a carrier. Advantageous carriers include absorbable pharmacologically acceptable solvents that serve to facilitate the passage through the skin of the host. Transdermal systems are usually in the form of a bandage that comprises a support, a supply container containing the active ingredient, if necessary together with carriers, optionally a separating device that releases the active ingredient onto the skin of the host at a controlled and established rate over a relatively long period of time, and means for securing the system to the skin.

Examples of formulations suitable for parenteral administration are aqueous solutions of an active ingredient in water-soluble form, for example in the form of a water-soluble salt, and also suspensions of active ingredient, such as corresponding oily injection suspensions, there being used suitable lipophilic solvents or vehicles, such as fatty oils, for example sesame oil, or synthetic fatty acid esters, for example ethyl oleate, or triglycerides, or aqueous injection suspensions that comprise viscosity-increasing substances, for example sodium carboxymethylcellulose, sorbitol and/or dextran, and, optionally, stabilizers. Dyes or pigments may be added to the pharmaceutical compositions, especially to the tablets or coatings, for example for identification purposes or to indicate different doses of active ingredient.

The pharmaceutical compositions that can be used for the present invention can be prepared in a known manner, e.g., by means of conventional mixing, granulating, confectioning, dissolving, coating or lyophilizing processes. For example, pharmaceutical compositions for oral administration can be obtained by combining the active ingredient with solid carriers, optionally granulating a resulting mixture, and processing the mixture or granules, if desired or necessary after the addition of suitable excipients, to form tablets, beads, pellets, or cores.

A suitable aromatase inhibitor can be administered at any time during a woman's menstrual cycle to practice the methods of this invention. For instance, it can be administered to the woman in need thereof on any day during the 3^{rd} to 22^{nd} day period of the cycle. On each day during the administration period, the number of dosages can be determined by the therapeutic need of the woman. For instance, up to 5 doses of aromatase inhibitor may be administered to a woman when a doctor determines that it is the appropriate number of dosages needed per day.

The appropriate amount of aromatase inhibitor in each dosage can also be determined by a doctor in view of the overall physical condition of the woman, the desired extent of endometrial thinning, the type of the aromatase inhibitor to be used, the safety and efficacy of the dose, and regimen in the population of women similarly situated. In general, it falls within the range of about 1 to 100 mg each dose, e.g., about 1 to 60, 1 to 30, or 1 to 15 mg per dose. Similarly, when a dose of aromatase inhibitor should be administered during a day can also be determined by a doctor in view of the woman's overall physical condition.

The efficacy of the methods of this invention may be determined by, e.g., measuring the thickness of the endometrium or the amount of bleeding in the woman undergoing the treatment. The thickness of the endometrium can be measured by techniques known in the art, e.g., by transvaginal ultrasound, SIS (saline infused sonohysterogram) or endometrial biopsies, while the amount of bleeding can be easily determined by visual determination.

In order that the invention described herein may be more fully understood, the following examples are provided. It should be understood that these examples are for illustrative purposes only and are not to be construed as limiting this invention in any manner.

### Example 1

This study is to establish whether there are differences in the proportion of women with thin endometria at the end of treatment based on when treatment is initiated compared to women not receiving active treatment.

Five different groups of pre-menopausal women are treated based on where there are in the menstrual cycle. Starting days (i.e., the first day of dosing) for the five groups are on Cycle Days 2-5, 6-9, 10-13, 14-17, and 18-21 for Groups 1-5, respectively.

A thin endometrium is defined as having a double-wall thickness not greater than 4 mm on transvaginal ultrasound. If anastrozole is first administered when the endometrium is thinnest at the start of the menstrual cycle, then it is likely that most women will maintain a thin endometrium throughout treatment (7-26 days). Conversely, when initiated after the mid-cycle when the endometrium will be thicker, suppression of estradiol with anastrozole is expected to induce a thinned endometrium at the end of treatment. The control group consists of a single group (similar size as a group of subjects receiving active treatment) of women taking placebo that are evaluated for endometrial thickness throughout the cycle on days that will match evaluation times for the five treated groups. The following table summarizes the dose day and evaluation times.

| | Dose | Cycle | Endometrial Thickness Assessments |
|---|---|---|---|
| | Day | Day | (Range of Cycle Days) |
| Group 1 | 1 | 2-5 | 5, 7, 11, 16, 20, 26 |
| Group 2 | 1 | 6-9 | 5, 7, 9, 11, 13, 16, 20, 26 |
| Group 3 | 1 | 10-13 | 10, 12, 14, 16, 18, 20, 23, 26 |
| Group 4 | 1 | 14-17 | 14, 16, 18, 20, 22, 24, 26 |
| Group 5 | 1 | 18-21 | 20, 22, 24, 26 |
| Control Group | 1 | 2 | 5, 7, 11, 14, 20, 26 |

The study is powered at the 0.80 level with a 0.10 significance level. It is assumed that the endometrial thickness for the controls will be considered as independent observations at each time they are obtained for comparison with an active treatment group. Based on these assumptions, a total of 6 subjects per group are able to detect an odds ratio of 7.5 between treated and untreated for an endometrial thickness ≦4 mm. Odds ratio for the above table equals 7.5 and would reject the hypothesis that there is no difference between treated and untreated for endometrial thickness less than 4 mm.

In addition to the evaluation at the end of the trial for total number of subjects with a thin endometrium, each group will be evaluated for mean endometrial thickness in order to determine if there are differences between active and non-active treatments. It is expected that subjects receiving anastrozole will have a thinner endometrium at the end of the treatment period compared to controls. It is also proposed that for the active treatment groups that endometrial thickness be assessed on the Treatment Days 1, 3, 5, and 7. In this way it will be possible to determine the trend in thickness within group. Women will also be asked to maintain a one week calendar of whether they experience any bleeding or spotting while receiving treatment. Other parameters will include laboratory measures of hormonal levels for estradiol, FSH, LH, in addition to normal safety laboratory measures.

This study is descriptive in nature and while there are criteria defined to provide guidance with regard to treatment timing, it is also intended to be able to explore relationships between and within the treatment groups to optimize the design of the ensuing Phase 2 dose ranging study. Minimally, it is expected at the completion of this study to be able to describe the appropriate time in which to initiate dosing to reduce estradiol levels and either induce or maintain a thin endometrium. It will also indicate whether thinning occurs rapidly after start of treatment or if there is time course in which to achieve thinning. Finally, the data will also indicate if there is anytime in the cycle in which treatment should not occur.

### Example 2

A study is conducted to determine the optimal dose for achieving a thinned endometrium when given at the time identified in Example 1. It is a randomized, placebo-control, double-blind, multi-center, dose ranging study that investigates the efficacy of anastrozole to suppress estradiol; and to thin the endometria of normal volunteer premenopausal women.

Women are screened during the appropriate cycle days prior to randomization, and the treatment is initiated within 3 days of screening. There are 4 treatment groups including placebo and 3 active treatment groups of 10 mg mg/day. The first dose is a “loading” dose and consists of two tablets on the first day of treatment. Days 2-7 (length of treatment differs depending upon results from Example 1 consist of a single tablet. Women recruited into the study are at least 25 years of age and with regular menstrual cycles, negative cytology and histology, appropriate sized uterus with no anatomic pathologies, non-pregnant, currently not using hormonal therapies or IUDs, with no known or suspected endocrine disorder and not receiving concomitant non-steroidal anti-inflammatory drugs. Women with a BMI greater then 35 kg/m², fibroids, and polyps are excluded.

Transvaginal ultrasounds and endometrial biopsies are performed during the screening period to assess for pathology. Treatment lasts for a total of 7 days (or less) and endometrial thickness is measured by the same ultrasound technician at each site on treatment days 1, 4, and 7. In addition, the myometrium is also measured at each of the clinic visits. Blood is drawn on the days in which ultrasound measurements are performed in order to assay for serum estradiol, FSH and LH.

The objective of this study is to demonstrate the most effective dose in suppressing estradiol and the correlative relationship with thinning the endometrium in pre-menopausal women. The primary outcome measure is estradiol suppression and published data with letrozole administered for 5 days provides an estimated standard deviation of approximately 4 pg/mL. In order to detect a difference of 8 pg/ml with α being 0.05 and power being 90%, a sample of size of 7 per group is needed.

Descriptive statistics is also obtained for endometrial thickness and the dose that has a significant difference from placebo and also produces (or maintains) a thin endometrium.

### Example 3

This study consists of a single placebo-controlled, randomized, multi-center clinical trial to determine the safety and efficacy of anastrozole at the dose and time identified in Examples 1 and 2 in thinning of the endometrium in women about to undergo an ablative procedure for persistent menorrhagia. Inclusion and exclusion criteria define a population of women with a history of menorrhagia that would be candidates for an ablative procedure of the endometrium. Pre-menopausal women are 20 years of age or older who are finished childbearing, candidate for endometrial ablation with regular menstrual cycles, negative cytology and histology, appropriate sized uterus with no anatomic pathologies, non-pregnant, desiring infertility, currently not using hormonal therapies or IUDs, with no known or suspected endocrine disorder and not receiving concomitant non-steroidal anti-inflammatory drugs were candidates. They also undergo follow-up for up to six months to determine differences in success rates between active treatments and control. The dose that achieves statistically and clinically meaningful differences from control is the dose for commercialization.

The dose of anastrozole is administered orally once a day and patients are instructed to be consistent with the time of day in which they take their randomized treatment. A loading dose (twice the dosage for the remainder of the trial) of anastrozole (or matching placebo) is administered at the time of randomization while a single dose follows for the remaining 6 (or less) days.

The sample size for the trial is based on the larger needed for the two primary endpoints. Using assumptions similar to that developed for gosrelin acetate, the sample size needed for detecting a 20% difference in amenorrhea between active and placebo six months after the ablative procedure, 136 patents per group is necessary or a total of 272 for the trial. The sample size calculated for endometrial thickness is 60 patients per group (total of 120 women) using a difference between active and placebo of 1.2 mm and a standard deviation of 2. Both the amenorrhea and thickness endpoints used a significance level of 0.05 and power of 90% to calculate the sample size.

The results of Examples 1-3 show that an aromatase inhibitor (e.g., anastrozole) can be administered at any time during a woman's menstrual cycle and an aromatase inhibitor (e.g., anastrozole) administered with a loading dose on the first day of dosing followed by a maintenance dose for 3-6 days is able to maintain a thin endometrium or to induce adequate thinning prior to ablation.

What is claimed is:

1. A method of thinning the endometrium in a woman, comprising administering to a woman in need thereof a therapeutically effective amount of an aromatase inhibitor.

2. The method of claim 1, wherein the aromatase inhibitor is administered to the woman at any time of the woman's menstrual cycle.

3. The method of claim 2, wherein the aromatase inhibitor is first administered on the 2^{nd}, 3^{rd}, 4^{th}, or 5^{th} day of the cycle.

4. The method of claim 2, wherein the aromatase inhibitor is first administered on the 6^{th}, 7^{th}, 8^{th}, or 9^{th} day of the cycle.

5. The method of claim 2, wherein the aromatase inhibitor is first administered on the 10^{th}, 11^{th}, 12^{th}, or 13^{th} day of the cycle.

6. The method of claim 2, wherein the aromatase inhibitor is first administered on the 14^{th}, 15^{th}, 16^{th}, or 17^{th} day of the cycle.

7. The method of claim 2, wherein the aromatase inhibitor is first administered on the 18^{th}, 19^{th}, 20^{th}, or 21^{st} day of the cycle.

8. The method of claim 3, wherein the aromatase inhibitor is administered once everyday for 2 to 10 consecutive days.

9. The method of claim 8, wherein the aromatase inhibitor is administered once everyday for 3 to 7 consecutive days.

10. The method of claim 8, wherein about 1 to about 300 mg of the aromatase inhibitor is administered on the first day.

11. The method of claim 10, wherein about 30 to about 300 mg of the aromatase inhibitor is administered on the first day.

12. The method of claim 11, wherein about 60 to about 150 mg of the aromatase inhibitor is administered on the first day.

13. The method of claim 10, wherein about 120 mg of anastrozole or about 60 letrozole is administered on the first day.

14. The method of claim 10, wherein about 1 to about 150 mg of the aromatase inhibitor is administered everyday after the first day.

15. The method of claim 14, wherein about 15 to about 150 mg of the aromatase inhibitor is administered everyday after the first day.

16. The method of claim 15, wherein about 30 to about 150 mg of the aromatase inhibitor is administered everyday after the first day.

17. The method of claim 15, wherein about 60 mg of anastrozole or about 30 mg of letrozole is administered everyday after the first day.

18. A method of preparing a woman for surgical ablation of the endometrium, comprising administering to the woman in need thereof a therapeutically effective amount of an aromatase inhibitor.

19. The method of claim 18, wherein the aromatase inhibitor is administered to the woman at any time of during the woman's menstrual cycle.

20. The method of claim 19, wherein the aromatase inhibitor is first administered on the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, or 21^{st} day of the cycle.

21. The method of claim 20, wherein the aromatase inhibitor is administered once everyday for 2 to 10 consecutive days.

22. The method of claim 21, wherein the aromatase inhibitor is administered once everyday for 3 to 7 consecutive days.

23. The method of claim 21, wherein about 1 to about 300 mg of the aromatase inhibitor is administered on the first day.

24. The method of claim 23, wherein about 30 to about 300 mg of the aromatase inhibitor is administered on the first day.

25. The method of claim 24, wherein about 60 to about 150 mg of the aromatase inhibitor is administered on the first day.

26. The method of claim 23, wherein about 120 mg of anastrozole or about 60 letrozole is administered on the first day.

27. The method of claim 23, wherein about 1 to 150 mg of the aromatase inhibitor is administered everyday after the first day.

28. The method of claim 27, wherein about 15 to 150 mg of the aromatase inhibitor is administered everyday after the first day.

29. The method of claim 27, wherein about 30 to 150 mg of the aromatase inhibitor is administered everyday after the first day.

30. The method of claim 27, wherein about 60 mg of anastrozole or about 30 mg of letrozole is administered everyday after the first day.

31. A method of treating menorrhagia in a woman, comprising administering to the woman in need thereof a therapeutically effective amount of an aromatase inhibitor.

32. The method of claim 31, wherein the aromatase inhibitor is administered to the woman at any time during the woman's menstrual cycle.

33. The method of claim 32, wherein the aromatase inhibitor is first administered on the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, or 21^{st} day of the cycle.

34. The method of claim 33, wherein the aromatase inhibitor is administered once everyday for 2 to 10 consecutive days.

35. The method of claim 34, wherein the aromatase inhibitor is administered once everyday for 3 to 7 consecutive days.

36. The method of claim 34, wherein about 1 to about 300 mg of the aromatase inhibitor is administered on the first day.

37. The method of claim 36, wherein about 30 to about 300 mg of the aromatase inhibitor is administered on the first day.

38. The method of claim 37, wherein about 60 to about 150 mg of the aromatase inhibitor is administered on the first day.

39. The method of claim 36, wherein about 120 mg of anastrozole or about 60 letrozole is administered on the first day.

40. The method of claim 36, wherein about 1 to 150 mg of the aromatase inhibitor is administered everyday after the first day.

41. The method of claim 40, wherein about 15 to 150 mg of the aromatase inhibitor is administered everyday after the first day.

42. The method of claim 40, wherein about 30 to 150 mg of the aromatase inhibitor is administered everyday after the first day.

43. The method of claim 40, wherein about 60 mg of anastrozole or about 30 mg of letrozole is administered everyday after the first day.

44. The method of claim 1, wherein the aromatase inhibitor is anastrozole, letrozole, exemestane, or combination thereof.
